(19) United States
(12) Patent Application Publication
(10) Pub. No.: US 2007/0249060 A1
(43) Pub. Date: Oct. 25, 2007

(54) Determination of Proteins and/or Other Molecules Using Mass Spectroscopy

(75) Inventors: Marc W. Kirschner, Newton, MA (US); Judith A. Jebanathirajah, Boston, MA (US); Muhammad N. Yousaf, Cambridge, MA (US)

Correspondence Address: WOLF GREENFIELD & SACKS, P.C., 600 ATLANTIC AVENUE, BOSTON, MA, 02210-2206, US

(73) Assignee: President and Fellows of Harvard College, Cambridge, MA (US)

(21) Appl. No.: 10/550,826

(22) PCT Filed: Mar. 26, 2004
(86) PCT No.: PCT/US04/09376

§ 371 Date: Dec. 13, 2006

Related U.S. Application Data
(60) Provisional application No. 60/458,105, filed on Mar. 26, 2003.

(51) Int. Cl. C07K 14/00; G01N 24/00

(52) U.S. Cl. 436/173

(57) ABSTRACT

This invention generally relates to the determination of species such as proteins and/or small molecules on self-assembled monolayers using mass spectrometry. In some cases, the proteins and/or small molecules may be arranged on a substrate in an array, for example, in a microarray. In one set of embodiments, the invention relates to methods for determining proteins and/or small molecules bound to self-assembled monolayers using mass spectroscopy techniques such as MALDI and MALDI TOF techniques. This combination allows, for example, the systematic identification of unknown proteins from cell lysates. Identification of novel interactions can be achieved, in some cases, in instances where the binding partner to a particular target species is unknown. In another set of embodiments, the invention relates to methods of attaching a species to a self-assembled monolayer on a substrate such that the substrate can be used in a mass spectrometer, in some cases without requiring additional exposure of the substrate to water. For example, a target species may be detected and/or analysed using mass spectrometry in the presence of similar or “contaminating” species, without first removing the contaminating species.

### RELATED APPLICATIONS

This application claims the benefit of U.S. Provisional Patent Application Ser. No. 60/458,105, filed Mar. 26, 2003, entitled “Detection of Interactions of Proteins and Other Molecules Using Mass Spectroscopy,” by Kirschner, et al, incorporated herein by reference.

### FEDERALLY SPONSORED RESEARCH

This invention was sponsored by the NIH, Grant Nos. R01GM39023 and R01HD37277. The Government may have certain rights to this invention.

### FIELD OF INVENTION

This invention generally relates to proteins and/or small molecules on self-assembled monolayers, and in particular, to the detection of proteins and/or small molecule on self-assembled monolayers using mass spectrometry.

### BACKGROUND

Protein microarrays are capable of analyzing thousands of samples in short time periods. However, protein microarrays are generally unable to systematically identify proteins within complexes of whole cell lysates, due to factors such as ill-defined coupling chemistry and nonspecific adsorption. It can also be difficult, in many cases, to identify proper binding partners for certain proteins within such lysates.

Protein microarrays often are printed on glass slides that are activated for binding. Typical methods for detecting proteins include ELISA detection and fluorescently-labeled antibodies, each of which can limit the detection of protein interactions to known proteins. Furthermore, the use of fluorescent-labeled antibodies to identify proteins may change or alter the nature of the protein interactions being studied, as the fluorescent label is typically an organic moiety that can interact in some fashion with the protein, changing or altering the protein

### SUMMARY OF INVENTION

This invention generally relates to the detection of proteins and/or small molecules on self-assembled monolayers using mass spectrometry. The subject matter of this application involves, in some cases, interrelated products, alternative solutions to a particular problem, and/or a plurality of different uses of a single system or article.

In one aspect, the present invention includes a method. In one set of embodiments, the method includes a step of determining, using mass spectrometry, a species suspected of being bound to a substrate having an array of entities bonded thereto, at least one of which the species is suspected of being able to bind to at least one of the entities.

In another set of embodiments, the method includes providing a substrate having an array of entities bonded thereto, allowing a species to bind to at least one of the entities, and without substantially desalting the substrate, applying mass spectrometry thereto.

In another aspect, the present invention includes an article. In one set of embodiments, the article includes a species bound to at least one of an array of entities on a substrate, wherein the species bound to the entity is detectable using mass spectrometry.

In another aspect, the present invention is directed to a method of making one or more of the embodiments described herein. In yet another aspect, the present invention is directed to a method of using one or more of the embodiments described herein. In still another aspect, the present invention is directed to a method of promoting one or more of the embodiments described herein.

Other advantages and novel features of the invention will become apparent from the following detailed description of various non-limiting embodiments of the invention when considered in conjunction with the accompanying figures. In cases where the present specification and a document incorporated by reference include conflicting disclosure, the present specification shall control.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIG. 1 illustrates one example method of the invention;

FIGS. 2A-2C illustrate an embodiment of the invention used to bind a protein in a cell lysate;

FIGS. 3A-3D illustrate an embodiment of the invention where single proteins are determined using mass spectrometry;

FIGS. 4A-4B illustrate an embodiment of the invention where a protein fragment is determined using mass spectrometry;

FIGS. 5A-5B illustrate an embodiment of the invention where a protein-protein interaction is determined using mass spectrometry;

FIGS. 6A-6B illustrate embodiments of the invention where a self-assembled monolayer is arrayed on a substrate in a microarray; and

FIGS. 7A-7B illustrate a self-assembled monolayer useful for certain embodiments of the invention.

### DETAILED DESCRIPTION

This invention generally relates to the determination of species such as proteins and/or small molecules on self-assembled monolayers using mass spectrometry. In some cases, the proteins and/or small molecules may be arranged on a substrate in an array, for example, in a microarray. In one set of embodiments, the invention relates to methods for determining proteins and/or small molecules bound to self-assembled monolayers using mass spectroscopy techniques such as MALDI and MALDI TOF techniques. This combination allows, for example, the systematic identification of unknown proteins from cell lysates. Identification of novel interactions can be achieved, in some cases, in instances where the binding partner to a particular target species is unknown. In another set of embodiments, the invention relates to methods of attaching a species to a self-assembled monolayer on a substrate such that the substrate can be used in a mass spectrometer, in some cases without requiring additional exposure of the substrate to water. For example, a target species may be detected and/or analyzed using mass spectrometry in the presence of similar or “contaminating” species, without first removing the contaminating species.

Each of the following is incorporated herein by reference in its entirety: U.S. Pat. No. 5,512,131, issued Apr. 30, 1996 to Kumar, et al.; International Patent Publication No. WO 96/29629, published Jun. 26, 1996, by Whitesides, et al.; International Patent Publication No. WO 99/54786, published Oct. 28, 1999, by Jackman, et al.; International Patent Publication No. WO 01/70389, published Sep. 27, 2001, by Ostuni, et al. U.S. Provisional Patent Application Ser. No. 60/458,105, filed Mar. 26, 2003, entitled “Detection of Interactions of Proteins and Other Molecules Using Mass Spectroscopy,” by Kirschner, et al, is also incorporated herein by reference.

The term “determining,” as used herein, generally refers to the analysis of a species, for example, quantitatively or qualitatively, and/or the detection of the presence or absence of the species. “Determining” may also refer to the analysis of an interaction between two or more species, for example, quantitatively or qualitatively, and/or by detecting the presence or absence of the interaction. In one aspect of the invention, the determination of a species includes using mass spectrometry, as further discussed below.

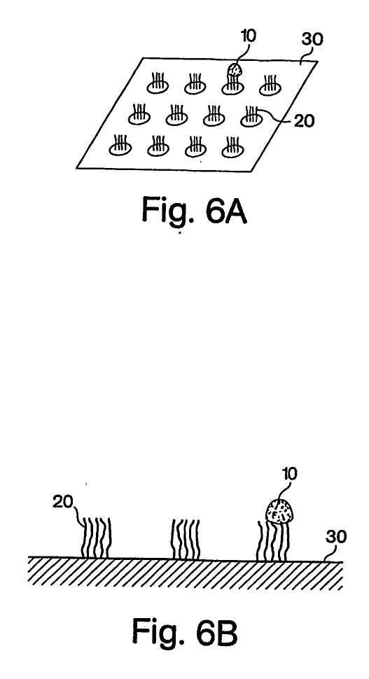

In one aspect of the present invention, a method is provided for determining species such as proteins and/or small molecules that may be bound to a substrate. For example, in FIGS. 6A and 6B, a species 10 is bound to a substrate 30, through a self-assembled monolayer 20 arrayed on the surface of the substrate. The binding may be specific or non-specific, depending on the application. In certain instances, the species is able to bind to a binding partner, as further described below. In some cases, the substrate may include a self-assembled monolayer, which may, for example, be used to array various proteins and/or small molecules on the substrate. Determination of the species may occur using any suitable technique, for example, mass spectroscopy techniques such as MALDI and MALDI TOF techniques.

As used herein, “specifically bound” or “adapted to be specifically bound” means a species is chemically or biochemically linked to or adapted to be linked to, respectively, another specimen or to a surface as described above with respect to the definitions of “fastened to,” “attached to,” “adapted to be fastened to,” and “adapted to be attached to,” but excluding essentially all non-specific binding. “Covalently fastened” means fastened via essentially nothing other than one or more covalent bonds. As one example, a species that is attached to a carboxylate-presenting alkyl thiol by essentially nothing other than one or more covalent bonds, which is, in turn, fastened to a gold-coated surface of a substrate, is covalently fastened to that substrate.

As used herein, a “protein” or a “peptide” is given its ordinary meaning in the art, i.e., a polymer comprising at least two amino acids. The protein or peptide may include amino acid sequences of at least about 5 amino acids, at least about 10 amino acids, at least about 50 amino acids, at least about 100 amino acids, or at least about 300 amino acids in some cases. In some instances, the protein or the peptide may include other entities besides amino acids, for example, carbohydrates or phosphate groups. An “amino acid” is given its ordinary meaning as used in the art, and may be a naturally-occurring amino acid (i.e., the 20 amino acids commonly found in nature) or an unnatural amino acid. An isolated amino acid typically, but not always (for example, as in the case of proline) has a general structure NH₂—CHR—COOH. In this structure, R may be any suitable moiety; for example, R may be a hydrogen atom, a methyl moiety, or an isopropyl moiety. A series of isolated amino acids may be connected to form a peptide or a protein, by reaction of the —NH₂ of one amino acid with the —COOH of another amino acid to form a peptide bond (—CO—NH—). In such cases, each of the R groups on the peptide or protein can be referred as an amino acid residue.

A “small molecule,” as used herein, means a molecule having a molecular weight of less than 5 kilodaltons (“kDa”), and more typically less than 1 kilodalton. A “dalton” (Da) is an alternate name for the unified atomic mass unit (grams/mole), and a “kilodalton” (kDa) is 1000 daltons. In some cases, the small molecule may be a protein or a peptide sequence. In other cases, however, the small molecule may be a member of any of a wide variety of organics; as non-limiting examples, the small molecule may be one or more of a carbohydrate, a sugar, a drug, an alcohol, a carboxylic acid, an amine, an aldehyde or a ketone, a thiol, a cyclic or an acyclic compound, etc.

In some cases, methods of the present invention may also be used to detect larger species (e.g., larger than a small molecule), for example, having molecular weights greater than about 1 kDa, greater than about 3 kDa, greater than about 5 kDa, greater than about 7 kDa, greater than about 10 kDa, greater than about 20 kDa, greater than about 30 kDa, greater than about 40 kDa, greater than about 50 kDa, greater than about 100 kDa, greater than about 200 kDa, or greater than about 350 kDa or more. Examples of such species include, but are not limited to, proteins, peptides, oligosaccharides, oligonucleotides, polymers, biopolymers, etc.

Species such as proteins and/or small molecules may be bound to a substrate through any suitable technique. For instance, the species may be bound to the substrate directly (i.e., a bond connects the species and the substrate) or indirectly (i.e., the species is bound to a “space” or a “linker,” which in turn is bound to the substrate, as further described below). In some cases, more than one spacer or linker may be present between the species and the substrate. A bond may be a chemical or a physical bond. Examples of bonds include, but not limited to, a covalent bond (which may be saturated or unsaturated), an ionic bond, a hydrogen bond, a van der Waals bond, a metal ligand bond, a dative bond, a coordinated bond, a hydrophobic interaction, or the like.

The characteristics of the substrate may vary widely, depending upon the intended use. The substrate may be formed in essentially any shape. In one set of embodiments, the substrate has at least one surface which is substantially planar. However, in other embodiments, the substrate may also include indentations, protuberances, steps, ridges, terraces and the like. In certain instances, the substrate can be non-planar, for example, in the form of a concave surface, a convex surface, a disc, a tubing, a cone, a sphere, and/or other geometric forms, or the like. In some cases, several substrate surfaces may be combined in some fashion. For example, two flat substrate surfaces may be joined together. In some cases, the two substrates may be integrally connected. As used herein, the term “integrally connected,” when referring to two or more objects, means that the objects cannot be separated manually, but requires at least the use of tools. In some cases, integrally connected parts cannot be separated without causing damage to at least one of the parts.

Suitable materials useful in the substrate include, but are not limited to, glasses, ceramics, plastics, metals, alloys, carbon, papers, agarose, silica, quartz, cellulose, polyacrylamide, polyamide, polyimide, and gelatin, as well as other polymer supports, other solid material supports, or flexible membrane supports. Polymers that may be used in the substrate include, but are not limited to, polystyrene, polytetrafluoroethylene (PTFE), polyvinylidenedifluoride, polycarbonate, polymethylmethacrylate, polyvinylethylene, polyethyleneimine, polyoxymethylene (POM), polyvinylphenol, polylactides, polymethacrylimide (PMI), polyalkenesulfone (PAS), polypropylene, polyethylene, polyhydroxyethylmethacrylate (HEMA), polydimethylsiloxane, polyacrylamide, polyimide, various block co-polymers, etc. The substrate may also comprise a combination of materials, optionally water-permeable, in some embodiments. In one embodiment, the substrate is disposable. In another embodiment, the substrate is reusable.

In some cases, the surface of the substrate may be modified in some fashion, for example, to create suitable reactive groups. Such reactive groups may include, for instance, simple chemical moieties such as amino, hydroxyl, carboxyl, carboxylate, aldehyde, ester, ether (e.g. thioether), amide, amine, nitrile, vinyl, sulfide, sulfonyl, phosphoryl, or similar chemically reactive groups. The reactive groups may also comprise more complex moieties including, but not limited to, maleimide, N-hydroxysuccinimide, sulfo-N-hydroxysuccinimide, nitrilotriacetic acid, activated hydroxyl, haloacetyl (e.g., bromoacetyl, iodoacetyl), activated carboxyl, hydrazide, epoxy, aziridine, sulfonylchloride, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, fluorobenzene, biotin and avidin. The reactive groups may also be, for instance, silanes, Si—OH, silicon oxide, silicon nitride, primary amines or aldehyde groups. Techniques of placing such reactive groups on a substrate by mechanical, physical, electrical or chemical methods are well known in the art. In some cases, the substrate may first be treated with reagents such as a strong acid before reactive groups are created on the surface. One of ordinary skill in the art will be able to readily identify suitable techniques for modifying the surface of the substrate as necessary for a particular application. Aldehyde groups may be useful in some embodiments to attach proteins to the substrate, as the aldehyde groups may react with N-termini of the proteins, thus allowing the proteins to interact with other proteins and/or small molecules in solution.

The substrate may also be conductive in some cases. Conductive substrates may be useful, for example, to facilitate desorption of species from the substrate, for example, during mass spectroscopy analysis of the species bound to the substrate. In some cases, a conductive substrate may allow the surface to be characterized using techniques such as, but not limited to, SEM, AFM, SIMS, GAFTIR, or the like

In certain embodiments, the substrate may be treated to block or inhibit non-specific binding, for example, between certain species such as proteins and/or small molecules and the surface of the substrate. In some cases, a buffer containing bovine serum albumin (BSA), casein, and/or nonfat milk may be applied to the substrate to block later non-specific binding between species and unreacted groups on the substrate.

In some cases, one or more species may be arranged on the surface of a substrate, forming an array. As used herein, an “array,” on a substrate, includes one or more discrete regions surrounded by a second region, for example, a first species may be arranged in one or more discrete regions on a substrate, surrounded by a region free of the first species. The discrete regions may each independently be the same or different, e.g., one region may contain a first species, while a second region may contain a first species and/or a second, different species, etc. The one or more discrete regions may be orderly arranged (for example, in a grid pattern) or not orderly arranged (for example, randomly distributed on the surface of the substrate). As an example, each of the species may be discretely arranged on the surface of the substrate, for example, forming a series of regions or “spots.” The spots may have any shape, for example, circular, oval, rectangular, square, arbitrary shapes, etc., and the arrangement of spots on the surface may be regular or irregular. The spots may each independently contain the same or different species, for example, one or more proteins, small molecules, other entities, etc. In some cases, at least some of the spots may have a maximum dimension of less than about 1 mm. Arrays having spots of such dimensions are also referred to herein as “microarrays.” In some cases, high density microarrays may be used, for example, with a density over about 10 spots/cm², over about 20 spots/cm², over about 30 spots/cm², over about 50 spots/cm², over about 75 spots/cm², over about 100 spots/cm², or over about 200 spots/cm². The arrays or microarrays of the invention may be produced by a number of means known to those of ordinary skill in the art. Such methods include, but are not limited to, microfluidics printing, microstamping (see, e.g., U.S. Pat. No. 5,515,131 and U.S. Pat. No. 5,731,152, each incorporated herein by reference), microcontact printing (see, e.g., PCT Publication WO 96/29629, incorporated herein by reference) and inkjet head printing.

If a substrate having an array of discrete spots is used, the spots may be maintained in some cases using an elastomeric membrane. As used herein, “elastomeric” defines an elastic polymer. The membrane (or similar articles) may be made of a polymeric material, and flexible polymeric materials are preferred in some embodiments of the invention. In some embodiments, the membrane comprises an elastomeric material. A variety of elastomeric materials may be suitable, especially polymers of the general classes of silicone polymers, epoxy polymers, and acrylate polymers. Epoxy polymers are characterized by the presence of a three-member cyclic ether group commonly referred to as an epoxy group, 1,2-epoxide, or oxirane. For example, diglycidyl ethers of bisphenol A may be used, in addition to compounds based on aromatic amine, triazine, and cycloaliphatic backbones. Another example includes the well-known Novolac polymers. Examples of silicone elastomers suitable for use as a membrane include those formed from precursors including the chlorosilanes such as methylchlorosilanes, ethylchlorosilanes, phenylchlorosilanes, and the like. One example of a useful silicone elastomer is polydimethylsiloxane. The membrane may also be reusable in some cases.

In some embodiments, linker molecules or “linkers” may optionally be added to the surface of the substrate to make the surface suitable for further attachment chemistry, for example, for attachment of self-assembled monolayers, as further discussed below. As used herein, the term “linker” means a chemical moiety which can immobilize a species with respect to the substrate, for example, by use of reactive groups on the substrate. As used herein, an entity that is “immobilized relative to” or “bonded to” another entity either is fastened to the other entity or is indirectly fastened to the other entity, e.g., by being fastened to a third entity to which the other entity is also fastened. For example, a species is immobilized relative to a substrate if an entity (such as a linker) fastened to the species attaches to the surface, where the species can be a single entity, a complex entity of multiple components, etc. In certain embodiments, an entity that is immobilized relative to another entity is immobilized using bonds that are stable, for example, in solution or suspension. In some cases, the entity may be immobilized relative to another entity using specific binding interactions.

Linkers may be selected from any suitable class of compounds and may comprise polymers or copolymers of organic acids, aldehydes, alcohols, thiols, amines and the like. In one aspect, the linker may be a self-assembled monolayer, as further described below. As additional examples, the linker may include polymers or copolymers of hydroxy-, amino-, or dicarboxylic acids, such as glycolic acid, lactic acid, sebacic acid, or sarcosine. Alternatively, polymers or copolymers of saturated or unsaturated hydrocarbons such as ethylene glycol, propylene glycol, saccharides, and the like may be employed as linkers in certain embodiments. In some cases, the linker may be of an appropriate length that allows a species to interact freely with molecules in a solution and to form effective binding.

Any suitable method may be used to bind or fasten a species to a linker or a substrate, such suitable methods being known in the art. As used herein, (“fastened to” or “attached to”) or (“adapted to be fastened to” or “adapted to be attached to”) as used in the context of an entity relative to another entity or an entity relative to a surface of an article (such as a substrate), means that the entity and/or surfaces are chemically or biochemically linked to or adapted to be linked to, respectively, each other via covalent attachment, attachment via specific biological binding (e.g., biotin/streptavidin), coordinative bonding such as chelate/metal binding, or the like. For example, “fastened” or “attached” in this context also includes multiple chemical linkages, multiple chemical/biological linkages, etc. As another example, a moiety covalently linked to a thiol is adapted to be fastened to a gold-coated surface since thiols are able to bind gold covalently. An entity also is adapted to be fastened to or attached to a surface if a surface carries a particular nucleotide sequence, and the species includes a complementary nucleotide sequence.

In some cases, the linker may include a self-assembled monolayer-forming entity. As used herein, the term “self-assembled monolayer” (“SAM”) refers to a relatively ordered assembly of molecules chemisorbed on a surface, in which the molecules are oriented approximately parallel to each other and roughly perpendicular to the surface. Each of the molecules includes a functional group that can be attached to the surface of the substrate, and a portion that interacts with neighboring molecules in the monolayer to form the relatively ordered array. Some of the methods that can be used to form a SAM are described in U.S. Pat. No. 5,620,850, which is hereby incorporated by reference. See also, for example, Laibinis, P. E., Hickman, J., Wrighton, M. S., Whitesides, G. M., Science, 245:845 (1989); Bain, C., Evall, J., Whitesides, G. M., J. Am. Chem. Soc., 111:7155-7164 (1989); Bain, C., Whitesides, G. M., J. Am. Chem. Soc., 111:7164-7175 (1989), each of which is incorporated herein by reference.

Certain embodiments of the invention make use of self-assembled monolayers attached to surface of the substrate. In one set of embodiments, SAMs formed essentially completely of synthetic molecules may be used in at least a portion of a substrate. “Synthetic molecule,” in this context, means a molecule that is not naturally occurring, rather, one synthesized under the direction of human or human-created or human-directed control. In some cases, the SAM can be made up of SAM-forming entity that form close-packed SAMs at surfaces, and/or these entities in combination with other entities able to participate in a SAM. In some embodiments, some of the entities that participate in the SAM include a functionality that binds, optionally covalently, to the surface, such as a thiol which will bind to a gold surface or a silver surface covalently. The SAMs may be characterized using various detection and identification methods known to those of ordinary skill in the art, for example, SPR, fluorescence microscopy, scintillation counting, phosphor imaging, and MALDI-mass spectrometry.

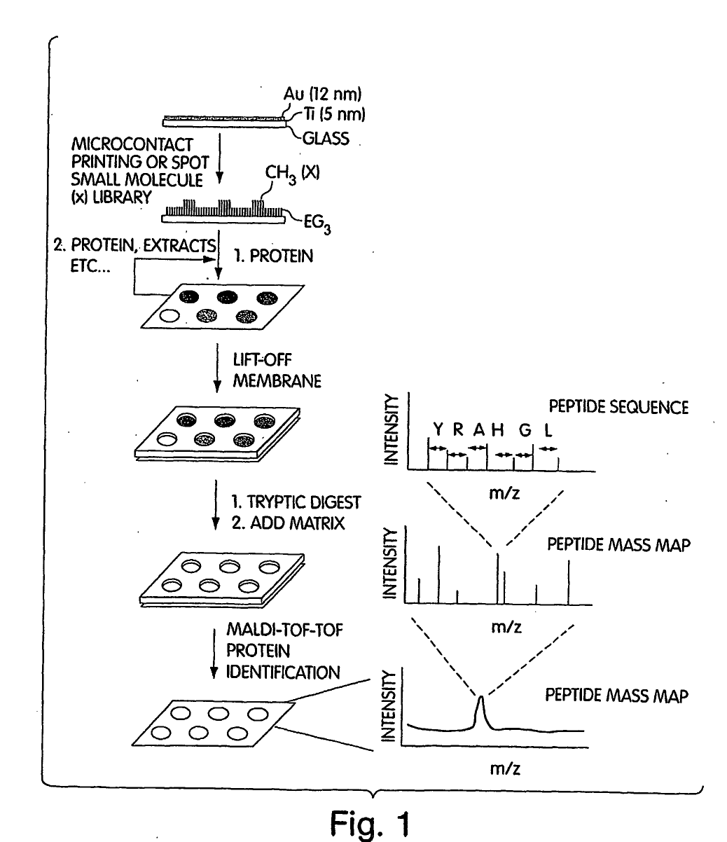

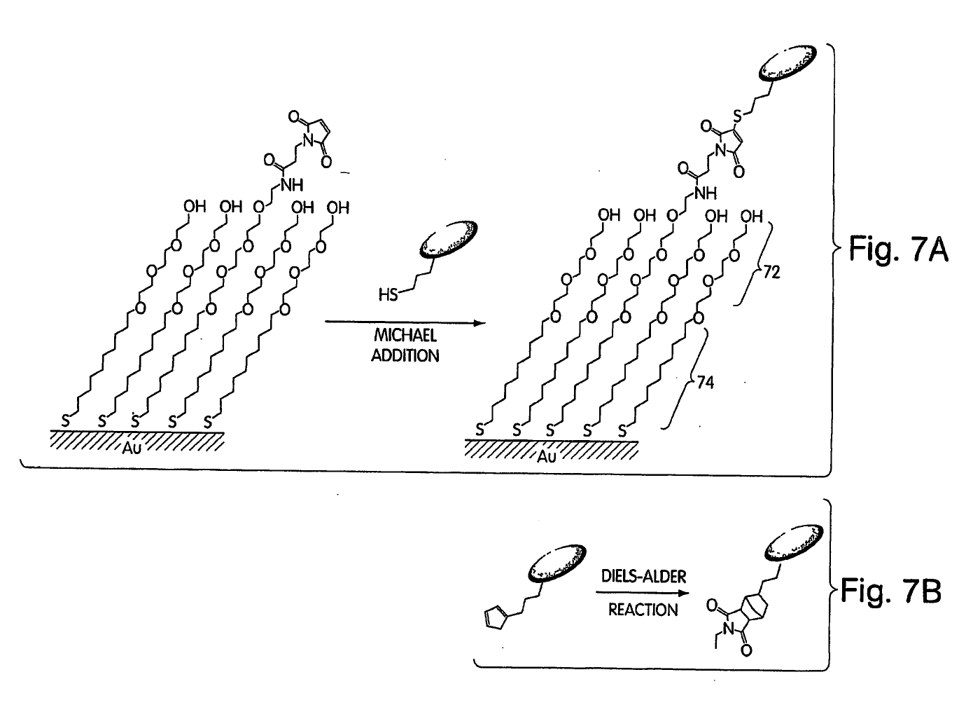

A self-assembled monolayer on a surface of a substrate, in accordance with an embodiment of the invention, may be comprised of a mixture of entities (e.g. one or more thiol species when the colloid has a gold surface) that can present (expose) essentially any chemical or biological functionality. For example, such entities can include triethylene glycol-terminated species (e.g. triethylene glycol-terminated thiols) to resist non-specific adsorption, and/or other entities, for example, thiols. In certain embodiments, SAMs on a surface may include an EG₃-terminated thiol, HS—(CH₂)₁₁—(O—CH₂—CH₂)₃—OH(“C₁₁EG₃”). EG₃-terminated SAMs on the surface may be able to resist the adsorption of proteins. In some cases, the EG₃-terminated SAMs surround regions that proteins will bind to, for example, as shown in FIG. 1. Another example of a SAM that resists the adsorption of proteins is shown in FIG. 7, where the oligo(ethylene glycol) chains 72 may prevent protein adsorption and the poly(methylene) chains 74 may give structural order.

In embodiments having species arranged on the surface of a substrate, the species arranged on the surface may be associated with a self-assembled monolayer (“SAM”), for example, in discrete spots. For example, one or more spots of the array can be defined by SAMs on a substrate. In some cases, a self-assembled monolayers may be defined by molecules each having a functional group that selectively attaches to a particular surface, the remainder of most or all of the molecules having characteristics allowing them to interact with neighboring molecules in the monolayer to form a relatively ordered array. The molecules may be generally largely or completely organic. Monolayers may be produced with varying characteristics and with various functional groups at the free end of the molecules which form the SAM (direction away from the surface to which the SAM attaches). Thus, SAMs may be formed which are generally hydrophobic or hydrophilic, generally cytophobic or cytophilic, or generally biophobic or biophilic. Additionally, SAMs with these or other characteristics can be formed and then modified to expose different functionalities. SAMs with very specific binding affinities can be produced in certain instances, which allows for the production of patterned SAMs which will bind one or more species on the surface in specific and predetermined patterns. A non-limiting example of micropatterned SAMs and their application is described in U.S. Pat. No. 5,776,748, issue Jul. 7, 1998, entitled “Method of Formation of Microstamped Patterns on Plates for Adhesion of Cells and other Biological Materials, Devices and Uses Therefor,” by Singhvi, et al., incorporated herein by reference.

In some cases, the species may be a binding partner. The term “binding partner” refers to a molecule that can undergo binding with a particular molecule. In some cases, the binding partner may be selected from molecular libraries, such as those commercially available. The binding partner may be bound to the substrate directly or the binding partner may be attached to a linker. The binding partner may be any of a variety of different types of naturally occurring or synthetic molecules, including those having biological significance (“biomolecules”). In some embodiments, the binding partner is a species as described above, e.g., a small molecule, a peptide, a protein, etc. As other examples, the binding partner may include naturally occurring molecules or molecule fragments such as nucleic acids, nucleic acid analogs (e.g., peptide nucleic acid), polysaccharides, phospholipids, capture proteins including glycoproteins, peptides, enzymes, cellular receptors, immunoglobulins, antigens, naturally occurring ligands, polymers, and antibodies (including antibody fragments) such as antigen-binding fragments (Fabs), Fab′ fragments, pepsin fragments (F(ab′)₂ fragments), scFv, Fv fragments, single-domain antibodies, dsFvs, Fd fragments, and diabodies, full-length polyclonal or monoclonal antibodies, and antibody-like fragments, such as modified fibronectin, CTL-A4, and T cell receptors.

Biological binding partners are examples of binding partners. For example, Protein A is a binding partner of the biological molecule IgG, and vice versa. The term “binding” refers to the interaction between a corresponding pair of molecules or surfaces that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones and the like. Specific non-limiting examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc.

Examples of methods of coupling the binding partner to the substrate or linker include reactions that form linkage such as thioether bonds, disulfide bonds, amide bonds, carbamate bonds, urea linkages, ester bonds, carbonate bonds, ether bonds, hydrazone linkages, Schiff-base linkages, and noncovalent linkages mediated by, for example, ionic or hydrophobic interactions. The form of reaction will depend, of course, upon the available reactive groups on both the substrate/linker and binding partner.

In some cases, a binding partner may be tagged in some fashion, for example, with fluorescent, radioactive, chromatic and other physical or chemical labels or epitopes. However, in other cases, the binding partner may be unlabeled, and detection may occur through the use of mass spectroscopy.

A wide variety of known mass spectrometry techniques may be used in the invention. Generally, a mass spectrometer is able to create charged particles (ions) from molecules (e.g., “ionization”). The ions are then accelerated by manipulation of the charged particles, while uncharged molecules and fragments are removed. For example, manipulation may occur through the manipulation of voltages to control the path of the ions, for instance, to direct the ions to a detector or a mass analyzer. Analysis of those ions may provide information about the original molecules, for example, molecular weight, structural information, or the like. There are many types of mass spectrometers and sample introduction techniques which allow a wide range of analyses, some of which are described below.

Non-limiting examples of ionization methods suitable for mass spectrometry that are known to those of ordinary skill in the art include, but are not limited to, electron impact, chemical ionization, electrospray, fast atom bombardment, and matrix-assisted laser desorption ionization (“MALDI”). MALDI may be useful in situations involving larger molecules, such as peptides, proteins, or nucleotides. In MALDI, the molecules may be identified by matching a list of molecular masses with a calculated list of all molecular masses, for example, from a database. In some cases, the ionized masses may be sequenced.

The ionized biomolecules are accelerated (typically using an electric field) towards a detector. During flight towards the detector, different molecules can be separated according to their mass to charge ratio and reach the detector at different times. In this way each molecule may produce a distinct signal. The method may be used for detection and characterization of species, including proteins and/or small molecules, as well as biomolecules such as those previously described.

Non-limiting examples of mass analyzers known to those of ordinary skill in the art include, but are not limited to, quadrupole, sector (magnetic and/or electrostatic), time-of-flight (TOF), or ion cyclotron resonance (ICR). In some cases, more than one mass spectroscopy technique may be used, and/or the mass spectroscopy technique may be combined with other techniques, for example, as in GC/MS, LC/MS, MS/MS, MALDI/MS, etc. Coupling two stages of mass analysis (e.g., as in MS/MS) may be useful in certain cases in identifying compounds in complex mixtures and in determining structures of unknown substances. For example, in certain such techniques, “parent” or “precursor” ions or mixture of ions in the source region or collected in an ion trap may be fragmented and then the product ions resulting from the fragmentation can then be analyzed in a second stage of mass analysis. Additional information for structural analysis may also be obtained from such techniques.

In some embodiments, the invention may be used to identify novel protein-protein interactions and/or protein-small molecule interactions in a high throughput fashion, for example using a tandem combination of MALDI MS and surface chemistry, as described above. According to one example embodiment, using self-assembled monolayers on gold may provide an easily adjustable surface for preparing and studying biointerfacial science using MALDI MS. This surface may allow for defined coupling chemistries, which may be used to eliminate positional ambiguities when a target is found to bind to a protein. Furthermore, the specific chemistry can be tailored in some cases to alleviate problems caused by non-specific binding and maintenance of the three-dimensional structure of the proteins attached.

The mass spectrometry technique is very sensitive in certain cases, i.e., the mass spectrometry technique may allow for the detection of low (10⁻¹⁵ mole to 10⁻¹⁸ mole) quantities of sample with an accuracy of about 0.1% to 0.01% in some cases. As an example, MALDI TOF (further described below) can be used to determine protein or peptide analysis and sequences, or provide information on microheterogeneity (e.g. glycosylation) and presence of by-products. In certain instances, the mass spectrometry technique can be used for the sequencing of nucleic acids.

In certain cases, the invention allows for the sequencing of molecules having higher molecular weights using mass spectroscopy techniques, for example, determining molecules having molecular weights greater than about 1 kDa, greater than about 3 kDa, greater than about 5 kDa, greater than about 7 kDa, greater than about 10 kDa, greater than about 20 kDa, greater than about 30 kDa, greater than about 40 kDa, greater than about 50 kDa, greater than about 100 kDa, greater than about 200 kDa, or greater than about 350 kDa or more. One non-limiting example of such a mass spectroscopy technique is MALDI TOF.

Matrix-assisted laser desorption/ionization-time of flight mass spectrometry (MALDI-TOF MS) is a technique in which a co-precipitate of an UV-light absorbing matrix and a biomolecule is irradiated by a short laser pulse, e.g., on the order of nanoseconds. A portion of the laser energy is absorbed by the matrix, which prevents unwanted fragmentation of the biomolecule. Those of ordinary skill in the art will be able to identify suitable materials for matrices for use with MALDI. Typically, the material is able to absorb ultraviolet light. Examples of such matrices include, but are not limited to, 2,5-dihydroxybenzoic acid, 3,5-dimethoxy-4-hydroxycinnamic acid (sinapinic acid), alpha-cyano-4-hydroxycinnamic acid, sinapinic acid, certain cinnamic acid derivatives, trans-cinnamic acid, etc.

Mass spectrometry techniques such as MALDI Tandem TOF MS can be employed for protein identification directly on the surface in certain cases. To identity proteins binding to targets on the substrate, an on-array (“on-chip”) digestion method may be used to produce peptides from the bound proteins in one aspect of the invention. In some cases, on-chip proteolytic digestion may occur without denaturing the proteins, and/or in the presence of salt. For example, in one set of embodiments, mass spectroscopy analysis of a substrate having one or more species attached thereon can occur without chromatographic purification (for example, as in an LC/MS technique). In some instances, the substrate may be used in mass spectrometry without substantially removing any salts present thereon. In some embodiments, one or more enzymes may be used for on-chip digestion, for example, but not limited to, trypsin, chymotrypsin, pepsin, etc.

Applications of the invention include, but are not limited to, determining proteins and/or small molecules, as well as determining protein-protein interactions and/or protein-small molecule interactions. In some cases, the proteins and/or small molecules to be determined may be in a mixture comprising multiple proteins and/or small molecules, for example, as would be present in a cell lysate. For example, if an array of the invention comprises antibodies, the array may first be exposed to a cell lysate, and various protein or other small molecules may be determined by then exposing the array to mass spectroscopy, as previously described. In some cases, the invention allows for the determination, using mass spectrometry, of a species suspected of being bound to a substrate in the presence of similar species which would interfere with determining the species in more conventional mass spectrometry techniques.

The following examples are intended to illustrate certain aspects of certain embodiments of the present invention, but do not exemplify the full scope of the invention:

### EXAMPLE 1

In this example, self-assembled monolayers were prepared on gold-coated glass slides and used to make spatially addressable protein and small molecule arrays. This example combines self-assembled monolayers on gold surfaces and MALDI tandem MS capabilities. This combination also allowed the identification of novel protein-protein and protein-small molecule interactions as described below. The arrays were incubated with cell lysates in order to capture binding partners to the proteins or small molecules. Lysates were prepared by homogenization in modified PBS buffer, 0.1% Triton X-100, with 1× protease inhibitor cocktail for tissue culture lysate (Sigma-Aldrich) and 400 micromolar Na₃VO₄, 20 mM NaF, and 2 mM sodium molybdate. Cell debris was removed by centrifugation.

An in situ digestion using various enzymes was performed on the arrays in some cases. For in situ digestion, N-octylglucopyranoside was added as a detergent to the array at a concentration of 10 mM. The array was then heated to a temperature of 70° C. The array was then cooled and a tryptic digest mixture was added to the surface such that the final concentration of trypsin of about 4.1 mg/ml in a 20 mM ammonium bicarbonate buffer at pH 7.8 was used. The sample was incubated for 5 hours in a 50° C. humid chamber. 5% formic acid was added to the surface to volatilize the ammonium bicarbonate and the array was dried in a vacuum chamber. The surface was then washed with 0.1% TFA and matrix was added. For whole protein MALDI, dihydroxybenzoic acid (DHB) was used in acetonitrile:water 1:2 (vol/vol) or sinapinic acid in acetonitrile:water 1:1 at a concentration of 10 mg/ml. For peptide MALDI, alpha-cyano-4-hydroxycinnamic acid matrix (5 mg/ml) was added to the chip and air-dried.

The arrays were also used as MALDI targets and interrogated using mass spectrometry. The mass spectrometers used in this example were a MALDI TOF, a Voyager DE STR (Applied Biosystems, Framingham, Mass.) and a MALDI TOF/TOF, ABI 4700 (Applied Biosystems). Each array was placed into the mass spectrometer such that the MALDI TOF mass spectrometer could be used as a detector for proteins bound to small molecules or proteins on the arrays. Intact proteins were detected using spots on these arrays as MALDI targets. This proved to be useful, for example, for screening combinatorial small molecule libraries for ligands using a particular protein. The programs Mascot (Matrix Science, London, UK), Protein Prospector and PepSea (Protana, Odense, DK) were then used to interrogate protein databases.

In order to use this system as discovery tool, i.e., to identify proteins from complex samples such as cell lysates, binding to e.g. antibodies, an efficient on-target digestion method without subsequent washing or chromatography steps was employed. The generated peptides were then sequenced/fragmented using a MALDI TOF/TOF tandem mass spectrometer, providing sufficient sequence information for unambiguous protein identification.

### EXAMPLE 2

This example describes the use of MALDI tandem TOF mass spectrometry for the identification of protein-protein interactions using a microarray. This method can also be termed “SPaM” for Self-assembled monolayers, in situ Proteolytic digestion and MALDI mass spectrometry to identify proteins on microarrays. This example also illustrates use of certain methods for the detection of proteins that interact with small molecules.

The identification of binding proteins was achieved by using an in situ microarray proteolytic digest along with MALDI tandem MS. The in situ digest produced peptides from binding proteins that were sequenced with high sensitivities, which allowed the unambiguous identification of protein binding partners. Slides were prepared using techniques similar to those described in Example 1. Surface treatments were used to reduce nonspecific surface interactions, as mass spectrometry is highly sensitive and nonspecific interactions tend to produce high backgrounds, as follows. SAMs were engineered to reduce nonspecific interactions by oligo(ethyleneglycol) groups interspersed with those presenting the molecule of choice at low densities were used on a gold surface. The SAMs included alkanethiolates, as alkanethiolates on gold may allow homogeneous complex monolayers comprising one, two or more alkanethiolates, the surfaces may be at least partially inert to non-specific protein adsorption, and the surfaces may be compatible in some cases with several detection and identification methods, for example, SPR, fluorescence microscopy, scintillation counting, phosphor imaging, and MALDI-mass spectrometry. Many surface techniques could also be used to characterize the surface, for example SEM, AFM, SIMS, GAFTIR, and the like. The use of the gold surface was also advantageous for MALDI tandem MS in this example, as the ionization and acceleration of analyte molecules required that a voltage be applied to the target. In some cases, the substrate was immersed in a solution of protein, which either adsorbs to the methyl-terminated regions or interacts specifically with the small molecule spotted regions of the monolayer. The arrays were then used to identify novel and known binding partners of the Anaphase Promoting Complex (APC).

In some cases, a complementary lift-off membrane was used. The lift-off membrane may isolate each spot of an array from other spots. Lift-off membranes were made hydrophilic via plasma oxidation to form a seal when placed on the substrate. Registry could be achieved by placement of the lift-off membrane on complementary patterned surfaces made from stamps having an inverse pattern.

To identify proteins in this example, an on-array digest compatible with MALDI ionization was developed. An on-target digestion (e.g., tryptic digestion) facilitated two methods of protein identification; by peptide mass mapping or by sequencing a peptide of interest. Both methods produced generally unambiguous protein identification, based on the number of proteins on the surface. Nonspecific interactions were also minimized to allow on-array target identification, as mass spectrometers are extremely sensitive and nonspecific interaction would tend to introduce a high background, thus suppressing peptide from real interactions.

In this example, a chemically flexible microarray platform was designed which allowed the immobilization of small molecules and/or proteins and which could be used as a discovery tool to identify proteins from a complex mixture binding to a particular ligand and/or novel protein interactions. In order to achieve this, several aspects of design were accounted for: reduction of nonspecific binding; developing a MALDI compatible in situ tryptic digest; engineering solutions to place the array into the mass spectrometer without compromising mass accuracy and resolution. MALDI TOF/TOF mass spectrometry was used in order to get unambiguous identification of the proteins based on peptide sequence information.

Using a MALDI TOF as a detector for the readout of the SAM-based arrays had several advantages. The gold surface was able to quench fluorescence, thereby limiting the sensitivity of detection by fluorescence. This permitted the analysis of binding studies on small molecule and/or protein arrays due to its sensitivity and its ability to function as a simple reader/detector as well as peptide sequencing tool, useful, for example, as a discovery tool for the analysis of unknown and novel interactions. As MALDI mass spectrometry is generally extremely sensitive, it was used to provide an inert surface to reduce and preferably eliminate false interactions that might take place. Certain gold surfaces were used that were selected to give specific interactions with biomolecules, for example, U.S. Pat. No. 5,776,748, incorporated herein by reference. The surfaces relied on self-assembled monolayers (SAM) that presented oligo(ethyleneglycol) groups.

A schematic is shown in FIG. 1. Substrates were prepared by first evaporating an adhesive titanium layer (about 3 nm) followed by gold (about 15 mm) on the surface of glass coverslips (18×18 mm, No. 2, Corning) in an electron beam evaporator. The evaporations were performed at a pressure of about 2×10¹⁷ Torr and at a rate of 0.5 nm/s for both metals. The gold-coated coverslips were washed with absolute ethanol before adsorption of alkanethiols.

Soft lithography, e.g., microcontact printing, was used to pattern either hexadecanethiol [HS(CH₂)₁₅CH₃] or small molecules terminated with alkanethiols onto the gold-coated surfaces. The patterns used in this example were round spots. The bare gold surfaces between the spots of alkylation were subsequently derivatized with a polyethylene glycol (PEG)-terminated alkanethiol [HS(CH₂)₁₁—(OCH₂CH₂)₃OH]. SAMs made from this alkanethiol provided generally inert surfaces that minimize nonspecific adsorption. A cotton swab wetted with a solution containing hexadecanethiol (2 mM in ethanol) was dragged once across the face of a polydithemylsiloxane stamp. The stamp was dried with a stream of nitrogen for about 30 seconds and gently placed on a gold coated glass slide with sufficient pressure to promote conformal contact between the stamp and the substrate. After about 20 seconds, the stamp was peeled from the substrate. The slide was immersed immediately in a solution of tetra(ethylene glycol) terminated alkanethiol (1 mM in ethanol) for 8-14 hours. The slide was removed, rinsed with absolute ethanol, and dried under a stream of nitrogen. Substrates were then immersed in a solution of protein for about 4 hours to adsorb protein to the regions of hexadecanethiolate.

As the spots were patterned in a registered and addressable manner, proteins of interest were then arrayed onto these patterns using a robotic spotter. The exposure of the hexadecanethiol substrate to a protein solution resulted in the formation of protein coated islands with a geometry and a distribution defined by the original SAM patterning. The optimal diameter of the arrayed spots for interrogation by MALDI TOF MS was experimentally determined to be 400 microns to 800 microns, separated by inert surfaces of approximately 1 mm to 2 mm. This distribution minimized spot to spot bleeding during the protein digestion and subsequent sample preparation steps.

Poly(dimethylsiloxane) (PDMS) was purchased from Dow Corning (Sylgard 184). PDMS stamps were prepared from photolithographically produced masters as described previously described in U.S. Pat. No. 5,512,131, incorporated herein by reference. Hexadecanethiol was purchased from Aldrich and purified by silica gel chromatography.

Instead of utilizing pure chemisorption, as described in this example, small molecules could also be arrayed using various chemistries for covalent immobilization such as the Michael addition (FIG. 7A) or the Diels-Alder reaction (FIG. 7B).

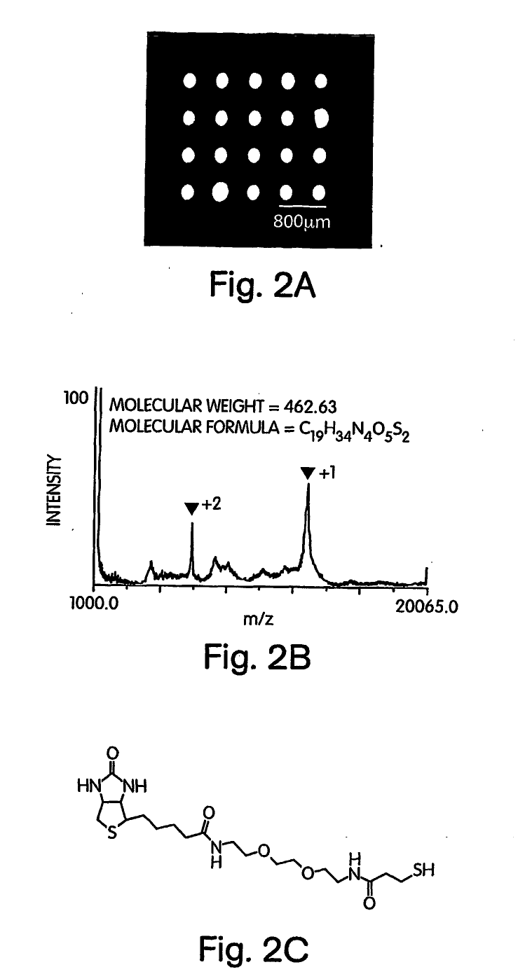

Once the small molecule or protein array was prepared, several subsequent experiments, each of which required different detectors/analytical methods for the read out, were performed. The chip was incubated (a) with a single protein, (b) with a mixture of known proteins, or (c) with a complex uncharacterized mixture such as whole cell lysate. After incubation with the protein solution of interest the array washed with the required stringency to remove buffer components and unbound proteins. For applications (a) and (b) the detection of the intact protein was performed (see below, discussion with respect to FIG. 2), whereas for application (c), the generation of proteolytic peptides by on-target digestion was performed to improve the sensitivity and to facilitate peptide sequencing. The former was accomplished by adding a MALDI matrix suitable for whole protein analysis, such as dihydroxybenzoic acid (DHB) or sinapinic acid (SA) onto the surface, and MALDI spectra was collected for the different spots on the array.

### EXAMPLE 3

In cases where peptides were utilized for the analysis in order to provide unambiguous protein identification, the problem of on-target digestion without any sample carry-over from one spot to the other was solved in this example as follows. The digest was performed in situ on the array in a humidity chamber without any prior reduction and alkylation. Subsequently, a MALDI matrix suitable for peptide analysis such as alpha-cyano-4-hydroxycinnamic acid was added to the digest array and the solution was evaporated under reduced pressure. The gold substrate with the array was then placed on a target that was specifically machined so as to accommodate the chip while maintaining the voltage on the surface of the chip. A gold coated array was useful for this type of analysis as the metal layer was conductive, thereby facilitating sensitive MALDI without unduly compromising accuracy and resolution.

In order to demonstrate this strategy with a small molecule-protein interaction, a surface was prepared with a SAM presenting a PEO-biotin (polyethylene oxide) terminated-thiol (FIG. 2C). The PEO-biotin used had an ethylene oxide linker which rendered the molecule hydrophilic and thus reduced nonspecific adsorption by proteins other than streptavidin.

The chip was rinsed, dried and incubated with a 2 pmol/ml solution of FITC coupled streptavidin. This was incubated overnight on a shaker and subsequently rinsed. The fluorescence image of the chip is shown in FIG. 2A. The image shows a generally clean distribution of the streptavidin protein on the PEO-biotin treated surface areas. Since the streptavidin binding sites are generally occupied with soluble biotin, regions where streptavidin is not found immobilized to the biotin indicate those regions where the surface is generally inert.

In a following experiment, a PEO-biotin chip incubated with streptavidin was overlaid on the chip with DHB as matrix at a concentration of 5 micrograms/microliters. The obtained MALDI TOF mass spectrum is shown in FIG. 2B. The spectrum is generally clean of any other nonspecifically adhering proteins present in the whole cell lysate. This demonstrates the feasibility of using this strategy to discover novel protein small molecule interactions. As this is a known protein of known mass, it was unnecessary in this particular example to perform a digest for more detailed analysis of the peptides.

### EXAMPLE 4

In this example, a single protein microarray for the SPaM strategy is demonstrated. Cytochrome C was applied to a SAM-based gold chip. Microcontact printing was used to generate surfaces patterned with hydrophobic circular regions (approximately 400 microns in diameter, 1.6 mm edge to edge distance). The protein array produced using techniques similar to those described above, and was incubated with a single protein in solution. The addition of 100 micrograms/ml of cytochrome C for 4 hours at 25° C. caused the adsorption of a single monolayer of protein to the hydrophobic regions of the microarray, as characterized by ellipsometry and SEM (data not shown). The ellipsometric film thickness measurements were made on a Gaertner Model L116C single-color optical ellipsometer equipped with a helium-neon laser operating at 632.8 nm, and interfaced to a personal computer. Measurements were made at an incident angle of 70°. The real and imaginary indices of refraction were measured at four or more locations on each substrate before monolayer formation. An average of these numbers was then inputted to the software to determine the thickness of the resulting film, which was also measured at four or more points. Reference optical constants for the bare gold film were measured for each substrate. An index of refraction of 1.46 was assumed for all independent determination of thickness and index of refraction.

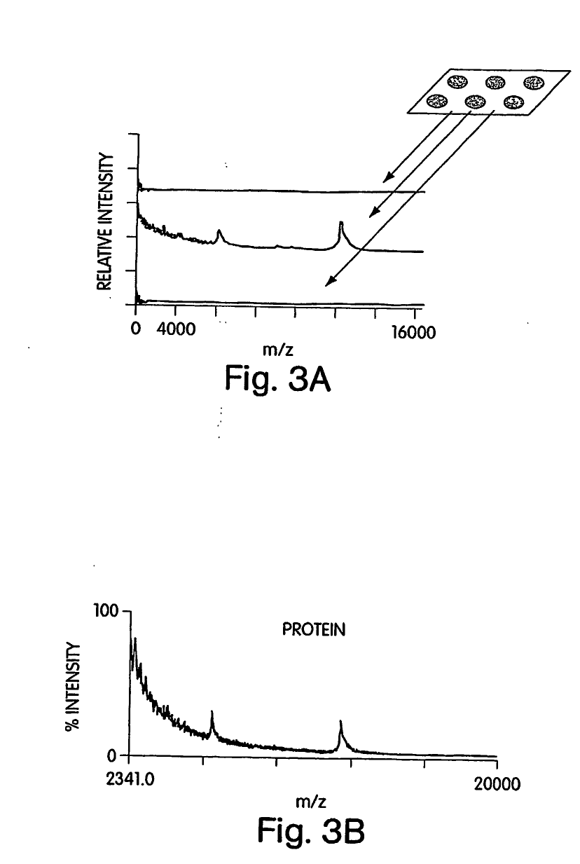

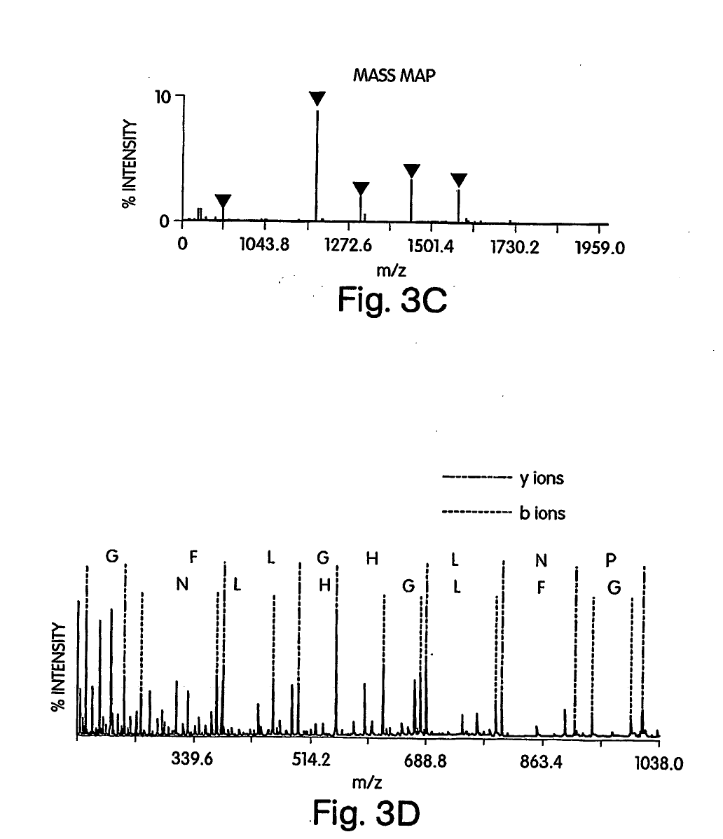

The array washed and MALDI matrix was applied to the array on the protein coated spot as well as on the gold surface derivatized with polyethylene glycol terminated alkanes, i.e. the surface areas inert to nonspecific protein binding. Spectra were collected by MALDI TOF MS on the spots as well as on the “inert” surface areas. The results of these experiments are shown in FIG. 3A. The inert surfaces were found to be generally clean of any protein whereas the spots with the physisorbed protein showed distinct signals for the singly, doubly and triply charged cytochrome C at 11.564 kDa, 5.782 kDa and 3.855 kDa. This type of analysis, i.e. using the MALDI TOF mass spectrometer as a simple “chip reader” in order to detect the protein of interest based on its mass, can also be utilized in cases where a single protein was being screened against a protein, peptide or small molecule library.

### EXAMPLE 5

If a complex protein mixture is used, the mass of the binding protein may not be sufficient for unambiguous identification. In these cases, an in situ digest on the target may also be performed in order to identify the protein by its peptide mass map or based on MS/MS data from one or several peptides. One example of this follows, where several conditions for in situ digestion were evaluated.

In the arrays used in this example, cytochrome C was immobilized onto various sized patterns (from 1 mm to 200 microns in diameter), and on chip in situ proteolytic digestion followed by MALDI-TOF MS analysis was performed. The following digestion conditions were studied in this example: low concentrations of ammonium bicarbonate (20 mM) as volatile buffer and increased temperatures of about 50° C. MALDI MS compatible detergents at low concentrations, such as 10 mM n-octyl-glucopyranoside (OGP) or acid labile detergents (RapiGest, Waters) were used for proteins in cases where the proteins were difficult to digest. After the digestion, 0.1% trifluoroacetic acid TFA was used as wash solution to remove ammonium bicarbonate buffer and excess detergent used to help the digest.

The peptides that resulted from the digest appeared to physisorb to the surface of the hydrophobic spot, as did the protein, thus allowing easy washing of the chip surfaces. The high affinity of the peptides to the hydrophobic areas also improved the sensitivity of the technique by concentrating the peptides to a small sample spot diameter in the sub-millimeter size range, which in turn reduced shot-to-shot heterogeneity, as often observed for traditional MALDI sample preparations. FIG. 3C shows the peptide mass fingerprint obtained after in situ digestion of cytochrome C. The digest produced numerous peptides with a sequence coverage of about 78%. After calibration using signals from trypsin autolysis peptides the spectrum was submitted for a MASCOT search, which unambiguously identified the protein as cytochrome C.

The feasibility of performing product ion experiment for sequencing purposes on a peptide on this surface at the peptide concentrations found on chip was also investigated in this example. MALDI MS/MS technology, including MALDI TOF/TOF and MALDI quadrupole/TOF technology, permitted the use of MALDI for the detection and identification of novel interactions. Several peptides were selected for fragmentation, and a spectrum from the precursor peptide at 1169.3 Da is shown in FIG. 3D. A clear sequence stretch covering most of the spectra collected were of sufficient quality to derive almost the entire peptide sequences, thus obtaining positive identifications using commonly used search algorithms such as MASCOT, Protein Prospecter and PepSea.

### EXAMPLE 6

As it was possible to perform in situ digests and to identify a protein bound to small molecules or physisorbed to these surfaces by peptide mass fingerprinting and also by fragmenting, i.e. sequencing a peptide, the following experiments established that the SAM-based arrays could also be used for the study of protein-protein interactions including mass spectrometric identification of the binding partners.

An anti-N-WASP antibody was physisorbed to a surface patterned with 400 micron spots, 1.6 mm edge to edge distance, by incubating the array with a solution of the anti-N-WASP antibody for 4 hours at 25° C. The surface washed in order remove excess antibody prior to incubation with 1 ml of HeLa whole cell lysate spiked with 200 ng (36 pmol) of recombinant N-WASP. The chip was subsequently extensively washed in PBS (pH 7.4) and dried under a stream of nitrogen, prior to overnight (about 12 hours) tryptic digestion on-target in situ.

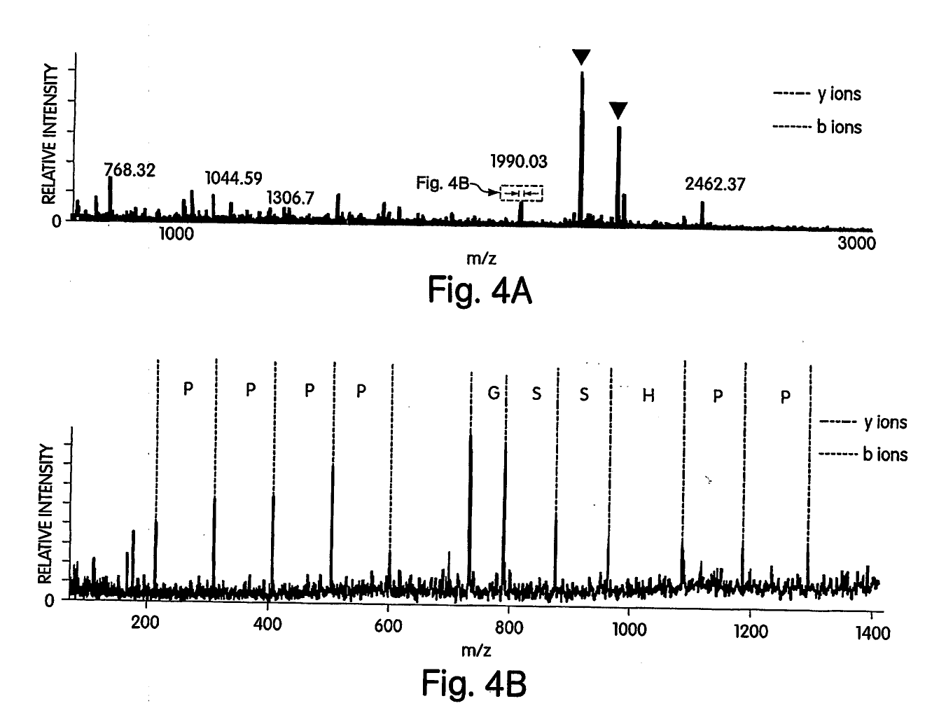

Matrix was then added to the surface and the surface was allowed to dry under reduced pressure. The MALDI TOF mass spectrum of this digest is shown in FIG. 4A. Apart from intense ion signals deriving from trypsin autolysis peptides and peptides deriving the antibody used for this experiment, several other peptide ion signals were found. These signals were used for a peptide mass fingerprint search after internal calibration utilizing the autolysis peaks as follows. MASCOT search software was used for the protein identification. The N-WASP was identified as top-scoring protein hit. As the MASCOT probability score did not provide an identification, several peptides were subjected to sequencing by MALDI tandem mass spectrometry to give a more clear identification.

The peptide at a m/z of 1990.2 was one of the peptides selected for sequencing by MS/MS. The acquired product ion spectrum is shown in FIG. 4B. This product ion spectrum was submitted to the MASCOT search engine and retrieved as top-scoring peptide a N-WASP-derived peptide with the sequence GGPPPPPPPHSSGPPPPPAR (SEQ ID NO: 1), thereby identifying the N-WASP protein. Other peptide signals observed in FIG. 4A include bovine trypsin at a m/z of 2162.049 and an IgG peptide at a m/z of 2172.1522. As with several product ion spectra deriving from the cytochrome c digest described above, this spectrum was of good quality for manual interpretation of peptide sequence stretches of up to 6 amino acid residues. To examine if a protein complex could be pulled down on the surface of the array an antibody to CDC27 was immobilized on the surface of the array.

### EXAMPLE 7

In this example, an anti-CDC27 antibody was immobilized on a SAM microarray in order to purify anaphase-promoting complex (APC). The SAM microarray was patterned with 400 micron diameter hydrophobic regions. CDC27 is a member of the well characterized anaphase promoting complex (APC). The array was then incubated with HeLa cell lysate at 25° C. and subsequently washed. An in situ digestion was carried out on the surface of the array and bound proteins from the lysate were identified using MALDI MS/MS. Known members of the anaphase-promoting complex, as well as novel binding partners, were then identified as follows.

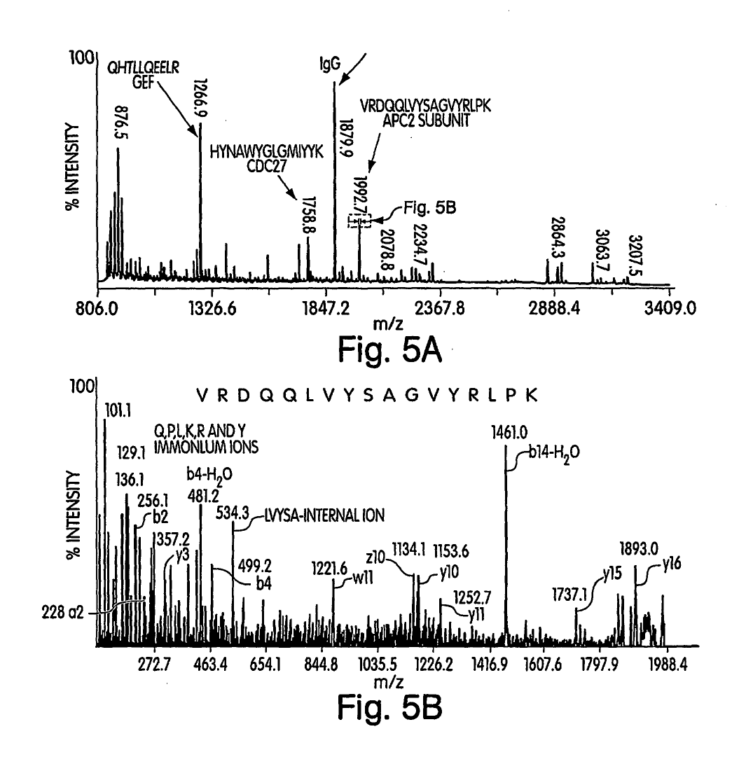

Alpha-cyano-4-hydroxycinnamic acid was added to the surface and spectra were collected, shown in FIG. 5. MALDI MS1 spectra are shown in FIG. 5A. One of the peptides, having a m/z of 1758.8, was sequenced and matched the peptide with the sequence HYNAWYGLGMIYYK (SEQ ID NO: 2), a peptide deriving from CDC27. A peptide having a m/z of 1992.7 was found to have a sequence VRDQQLVYSAGVYRLPK (SEQ ID NO: 3), and was identified as an APC2 peptide, another subunit of APC. A peptide having a m/z of 1266.9 was found to have a sequence QHTLLQEELR (SEQ ID NO: 4), and was identified as GTP exchange factor peptide (GEF). GTP exchange factor has several distinct domains which may indicate that the protein is a substrate of APC. Some of the peptides on the surface were then fragmented and the data interrogated using Protein Prospector, MS-Tag, Mascot, and Pepsea. FIG. 2B shows an example spectra which identified a peptide from APC subunit 2, an integral protein with Cullin homology.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

It should also be understood that, unless clearly indicated to the contrary, in any methods claimed herein that include more than one act, the order of the acts of the method is not necessarily limited to the order in which the acts of the method are recited.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “only one of” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1-33. (canceled)

34. A method of mass spectrometry, comprising:
exposing a sample containing a protein to a substrate;
allowing the protein to become immobilized with respect to the substrate;
exposing the protein immobilized with respect to the substrate to mass spectral ionization conditions to generate ionized fragments;
determining, via analysis of the identity of the ionized fragments, the identity of the protein.

35. A method as in claim 34, wherein the sample contains a mixture of proteins, the method comprising, prior to exposing and determining:
exposing the sample containing a mixture of proteins to a substrate;
allowing the at least one protein to become immobilized with respect to the substrate; and
removing any non-bound protein from the substrate.

36. The method of claim 34, wherein the exposing step comprises exposing the protein immobilized to mass spectral ionization conditions using MALDI mass spectrometry.

37. The method of claim 36, wherein the exposing step comprises exposing the protein immobilized to mass spectral ionization conditions using MALDI TOF mass spectrometry.

38. The method of claim 34, wherein the determining step comprises determining the species using a first mass spectrometry technique and a second mass spectrometry technique.

39. The method of claim 34, wherein the determining step comprises determining at least a portion of a primary sequence of the protein.

40. The method of claim 34, wherein the protein is part of a mixture of substances.

41. The method of claim 40, wherein the mixture comprises cell lysate.

42. The method of claim 34, wherein the substrate includes a microarray.

43. The method of claim 34, wherein the substrate includes a protein array.

44. The method of claim 34, wherein the substrate includes a self-assembled monolayer.

45. The method of claim 34, wherein the substrate includes a gold surface.

46. The method of claim 34, wherein the protein is able to specifically bind to an entity that is bonded to the substrate.

47. The method of claim 46, wherein the entity is an antibody.

48. The method of claim 46, wherein each entity is individually addressable by mass spectrometry.

49. A method of mass spectrometry, comprising:
exposing a mixture of proteins to a substrate;
allowing at least one protein to become immobilized with respect to the substrate;
removing any non-bound protein from the substrate;
exposing the at least one protein immobilized with respect to the substrate to an ionization method to generate ionized fragments; and
determining, via mass spectrometry of the identity of the ionized fragments, the identity of the at least one protein.


SEQUENCE LISTING (4 sequences)

SEQ ID NO: 1 - protein, 20 aa, Artificial sequence
  N-WASP fragment
GGPPPPPPPHSSGPPPPPAR

SEQ ID NO: 2 - protein, 14 aa, Artificial Sequence
  CDC27-derived fragment
HYNAWYGLGMIYYK

SEQ ID NO: 3 - protein, 17 aa, Artificial Sequence
  APC2 peptide
VRDQQLVYSAGVYRLPK

SEQ ID NO: 4 - protein, 10 aa, Artificial Sequence
  GTP exchange factor peptide
QHTLLQEELR